(19) United States
(12) Patent Application Publication
(10) Pub. No.: US 2022/0249806 A1
(43) Pub. Date: Aug. 11, 2022

(54) MAGNETIC SYSTEM FOR REMOTE CONTROL OF OBJECTS IN A BIOLOGICAL LUMEN

(71) Applicants: Michael SHPIGELMACHER, Los Angeles, CA (US); Alexander SROMIN, Ashdod (IL); BIONAUT LABS LTD., Herzliya (IL)

(72) Inventors: Michael SHPIGELMACHER, Los Angeles, CA (US); Alexander SROMIN, Ashdod (IL)

(73) Assignee: BIONAUT LABS LTD., Herzliya (IL)

(21) Appl. No.: 17/617,355

(22) PCT Filed: Jun. 10, 2020
(86) PCT No.: PCT/US20/37012

§ 371 Date: Dec. 8, 2021

Related U.S. Application Data
(60) Provisional application No. 62/859,824, filed on Jun. 11, 2019.

(51) Int. Cl. A61M 25/01; A61B 34/00; A61M 31/00; B03C 1/033; B03C 1/28

(52) U.S. Cl. CPC A61M 25/0127; A61B 34/73; A61M 25/0116; A61B 2017/00876; B03C 1/0332; B03C 1/288; A61M 31/002

(57) ABSTRACT

Remotely-controllable internal devices for insertion into a biological lumen and similar spaces within biological organisms; and magnetic systems for remote control thereof. Embodiments include mechanisms for linear motion within a lumen, mechanisms for payload release of therapeutic, diagnostic, and examination materials, and anchoring mechanisms for affixing a device to the outer wall of a lumen for a variety of medical and biological purposes. Related embodiments provide additional features including gradual payload release and reversibly-anchoring mechanisms Different functionalities (motion, payload release, anchoring) are independently-controllable through embodiment configurations featuring differing magnetic force thresholds and orthogonally-oriented magnetic responses.

### FIELD

The present invention relates to the use of externally-generated magnetic fields for remote control of internal devices inserted within a biological matrix.

### BACKGROUND

In medical applications, it is often necessary to control the motion and/or the mechanical operation of an internal device inserted within a biological matrix, a non-limiting example of which is a “lumen.”

Medical uses of internal devices include, but are not limited to: therapeutic, surgical, and diagnostic actions, as well as any sequence or combination thereof. Non-limiting examples of internal devices which may need to be controlled in this matter include: stents, catheters, micro-robots, micro-pumps, “smart pills,” fiduciary markers for imaging, sensors, and radioactive plaque.

A major challenge in the field is attaining remote control of the motion and operation of an internal device in a robust, versatile, reliable, efficient, cost-effective, and safe manner. This goal is achieved by embodiments of the present invention.

### SUMMARY

Embodiments of the present invention provide remotely-applied magnetic fields and remote-control mechanisms allowing wireless control of internal device motion and mechanical operation within a lumen or similar region within a biological matrix. Although embodiments disclosed herein are described with reference to lumens, it is understood that such descriptions relating to lumens are non-limiting, and that remotely controlling internal devices within other features of a biological matrix are also covered by alternative embodiments, where applicable.

Terms used herein:
The term “biological matrix” herein denotes a biological specimen or portion thereof, including, but not limited to: biological organisms regardless of species, whether living or dead, and whether active, sedated, or anesthetized (and such a biological organism under medical examination, diagnosis, and/or treatment is herein also denoted as a “patient”); biological organs, tissue, embryonic matter, and the like, occupying a geometric region and having a boundary separating one or more internal spaces from spaces external thereto.
The term “lumen” herein denotes a biological channel schematically described as an interior cavity of a tubular organ or similar construct having a surrounding outer wall or surface. Non-limiting examples of biological structures having lumens include: blood vessels, the sub-arachnoid space, and biliary ducts.
The term “outer wall” herein denotes tubular or similarly-disposed tissue surrounding a lumen (as in “the outer wall of a lumen”; in a non-limiting example, the tissue of an artery is herein referred to as “the outer wall of the artery's lumen”); or, depending on context, “outer wall” herein also refers to an inner surface of tubular or similarly-disposed tissue surrounding the lumen.
   It is not necessary that the outer wall tissue surrounding a lumen be uniform, and distinct “outer wall sides” may be separately identified in those cases where the tissue is non-uniform and varies from place to place within a lumen.
The term “internal device” herein denotes a manufactured construct foreign to the biological matrix, which is inserted therein and is remotely controllable from a region external to the biological matrix; and which denotes such a construct regardless of the materials or fabrication thereof, whether of mechanical or molecular construction, and whether created by assembly from components, by printing, by synthesis, or by other physical and/or chemical and/or biological means.
The term “external,” when appearing without further qualification, herein denotes a region outside the boundaries of a particular biological matrix, and when used as an adjective, denotes its modified noun as being located in such a region.

Therefore, according to an embodiment of the present invention, there is provided: (a) a device for magnetically controlling a target object within a lumen, the device including: (b) at least two permanent dipole magnets of like dimensions and corresponding like faces, and having like poles disposed on respective like faces thereof; (c) a yoke incorporating a material capable of completing a magnetic circuit; (d) wherein the at least two permanent dipole magnets are affixed to the yoke adjacent to one another such that: (e) like pole faces of the at least two permanent dipole magnets are affixed in contact with a same single face of the yoke; (f) the at least two permanent dipole magnets are affixed to the yoke in locations on the same single face of the yoke such that an inter-magnet space separates the at least two permanent dipole magnets; and (g) respective opposite pole faces of the at least two permanent dipole magnets are exposed, adjacent, and separated by the inter-magnet space.

According to another embodiment of the present invention, there is provided: an internal device for implanting into a biological medium and for responding to control by an external magnetic control system, the internal device comprising a plurality of linearly-interconnected magnetic elements along a device axis, such that each magnetic element of the plurality is adjacently-connected to at least one other element and at most two other elements, wherein: each pair of adjacent magnetic elements is interconnected by a non-magnetic flexible connector; and each magnetic element of the plurality has at least one predetermined function selected from a group consisting of: a function relating to control by the external magnetic control system; and a function relating to data communication with the external magnetic control system.

### BRIEF DESCRIPTION OF THE DRAWINGS

The subject matter disclosed may best be understood by reference to the following detailed description when read with the accompanying drawings in which:

FIG. 1 schematically illustrates a lumen having distinct outer wall sides, and an internal device therein controlled by an external magnet.

FIG. 2 through FIG. 8 illustrate various configurations of an external magnetic system according to embodiments of the present invention.

FIG. 9 illustrates a cross-section (x-z plane) of a lumen and internal device locations relative to an external magnet, according to an embodiment of the invention.

FIG. 10 illustrates adjusting motions for external magnets, according to an embodiment of the invention.

FIG. 11A through FIG. 11D illustrate mechanisms for payload release from an internal device according to embodiments of the present invention.

FIG. 12 illustrates a mechanism for anchoring an internal device to an outer wall of a lumen according to embodiments of the present invention.

FIG. 13 illustrates an internal device, according to embodiments of the present invention, with two magnetic components connected by non-magnetic flexible connectors to a payload release element in between them.

FIG. 14 illustrates a moving internal device that reverses direction upon reaching the target according to embodiments of the present invention.

FIG. 15 depicts an internal device, according to embodiments of the present invention, with two magnetic components and a cavity in between them.

FIG. 16 shows the distribution of equilibrium force and equilibrium angle, for an embodiment of the magnetic system corresponding to FIG. 8.

FIG. 17A through FIG. 17E depicts various configurations of an internal device according to embodiments of the present invention.

For simplicity and clarity of illustration, elements shown in the figures are not drawn to scale, and the dimensions of some elements may be exaggerated relative to other elements. In addition, dimensions illustrated and/or disclosed in the text are exemplary and non-limiting, and typically vary from one embodiment of the invention to another.

### DETAILED DESCRIPTION

An internal device is initially introduced into a lumen via an insertion process. Insertion mechanisms are well-known, and include, but are not limited to: injection and ingestion. After insertion into the lumen, it is desired to control the motion and mechanical operation of an internal device remotely in a manner having the qualities previously noted.

In some applications, it is desired that an internal device moves along a specific outer wall side of a lumen for anatomical reasons. In a non-limiting example, within the sub-arachnoid-space, one side of the lumen is the Dura mater, and another side is the Pia mater. The Pia mater is more delicate than the Dura mater, and it is therefore desirable for internal devices within the sub-arachnoid lumen to remain proximate to the outer wall side associated with the Dura mater and to avoid coming close to or having contact with the outer wall side associated with the Pia mater. In discussions relating to certain embodiments of the invention, there is a “preferred outer wall side” of the lumen denoted as “side A,” with an opposite outer wall side denoted as “side B.”

In certain embodiments, a magnetic component (such as a permanent magnet or ferromagnetic material) is embedded in an internal device.

According to these embodiments, in order to maintain the internal device closer to side A, an external magnetic device is positioned outside of the biological matrix, closer to side A than to side B. The external magnetic device attracts the internal device, keeping it closer to side A.

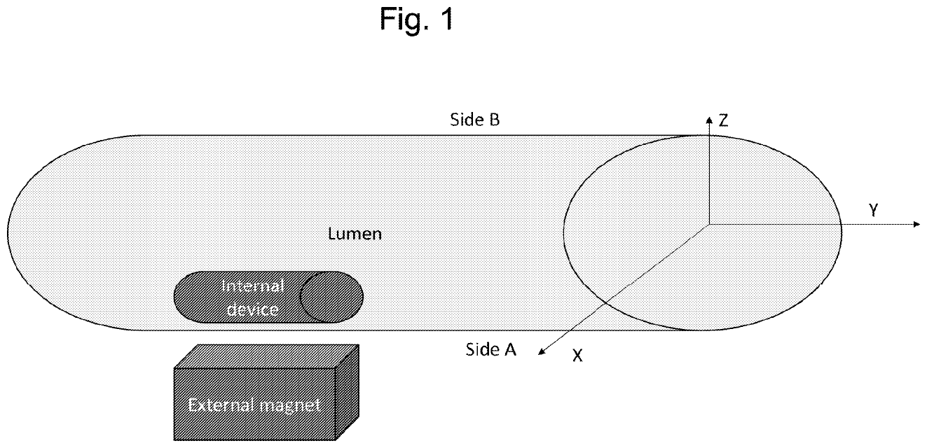

As illustrated in FIG. 1, in the case of a lumen which approximates a straight line over a region, and where an external magnet is brought as close to the lumen as possible to the outer wall, then by moving the external magnet in the direction along side A of the lumen, the magnetic interaction causes the internal device to move along side A, thereby controlling the motion of the internal device.

However, it should be noted that in real situations, various complicating factors typically exist:
The lumen may be curved.
The distance between the lumen and the external magnet may be bounded from below due to anatomical constraints (since the external magnet is located outside the biological matrix, it cannot be brought arbitrarily close to the lumen).
The outer wall of the lumen may be irregular or may adhere to the internal device, impeding smooth sliding motion of the internal device.
The outer wall of the lumen may contain specific biological or anatomical features, contact with which the internal device should avoid for medical reasons (e.g., a particular blood vessel or a neural bundle).
The lumen may be discontinuous or may contain bifurcations.
The aspect ratio of the internal device (length/width) may be comparable to the smallest dimension of the lumen, in consequence of which certain spatial orientations of the internal device may cause it to become stuck, thereby blocking the lumen and rendering further motion difficult or impossible.

In a further embodiment of the invention, the above restrictions are overcome to allow for internal device movement along curved trajectories in a lumen, accounting for irregularities and/or adhesion of an outer wall, providing for movement along different lumen bifurcations, accounting for non-zero distances between an internal device and an external magnet, and providing for control of internal device spatial orientation within a lumen.

This further embodiment relies on the following features:
The internal device contains an embedded magnetic component having a predefined N-S magnetization direction.
The external magnetic system includes multiple permanent magnets assembled in a predetermined spatial configuration. A related embodiment provides an additional ferromagnetic yoke (in a non-limiting example, made of iron or magnetic stainless steel).
The combination of the magnetization vectors of the different components of the external magnetic system generates a combined force vector and a combined torque vector on the internal device, allowing control of both the direction of motion of the internal device as well as its three-dimensional spatial orientation.

In a non-limiting example, a lumen is the sub arachnoid space, located at a distance of approximately 30 mm from the skin surface. For non-limiting exemplary purposes, an internal device is cylindrical with a diameter of 0.8 mm and a height of 0.8 mm.

### Motion in Y-Z Plane

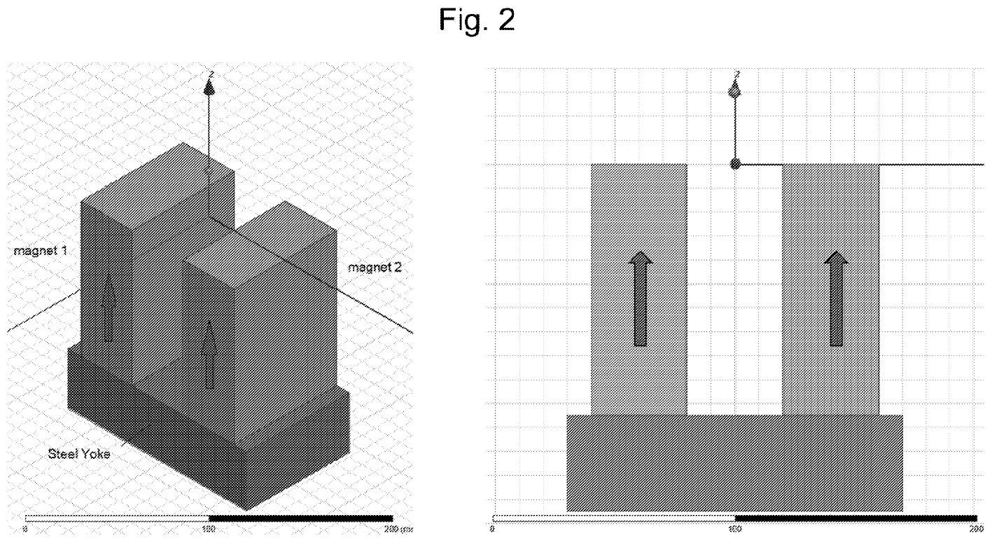

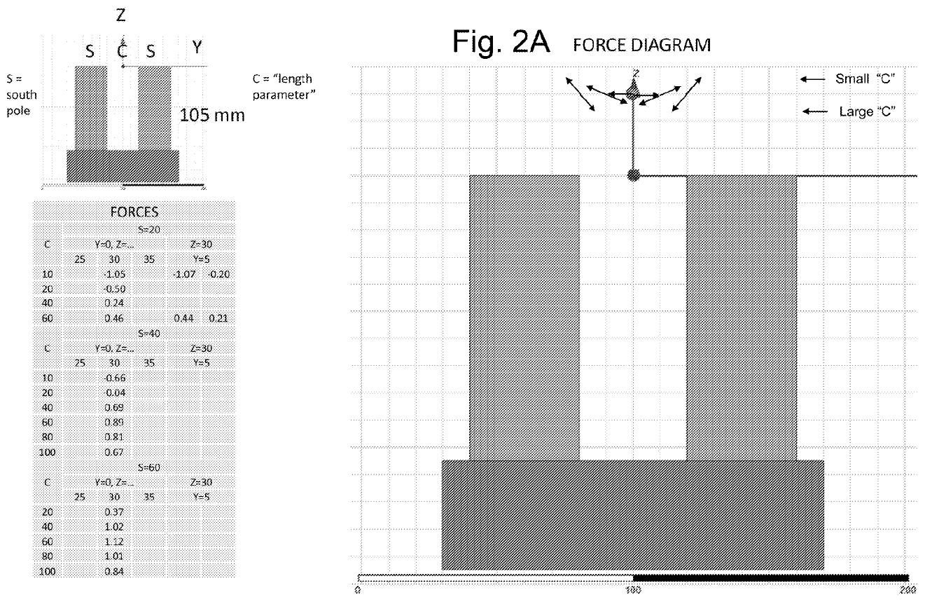

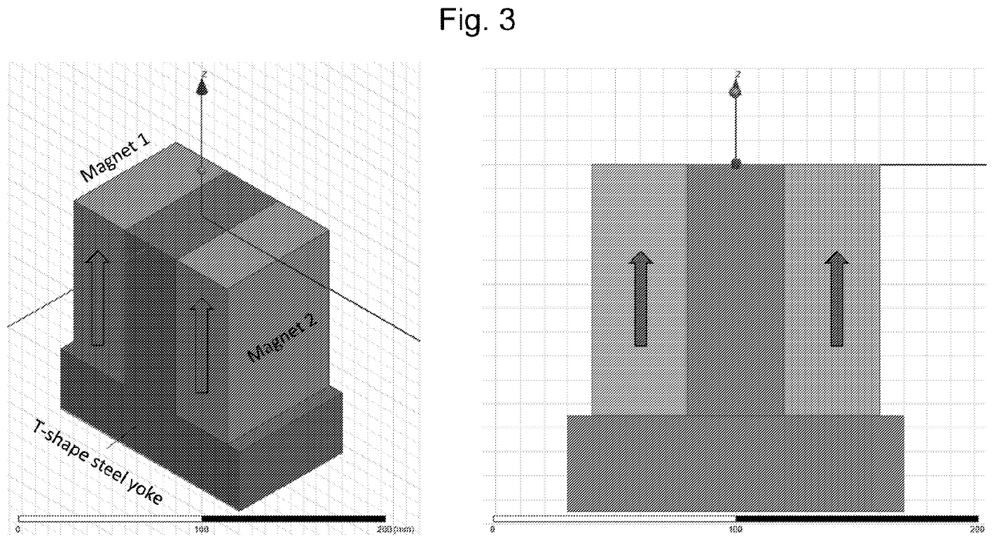

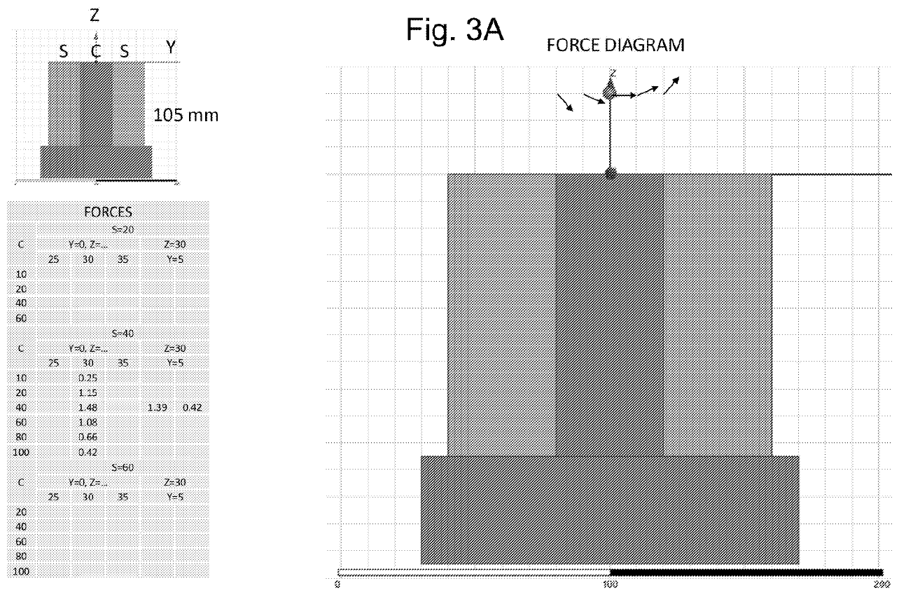

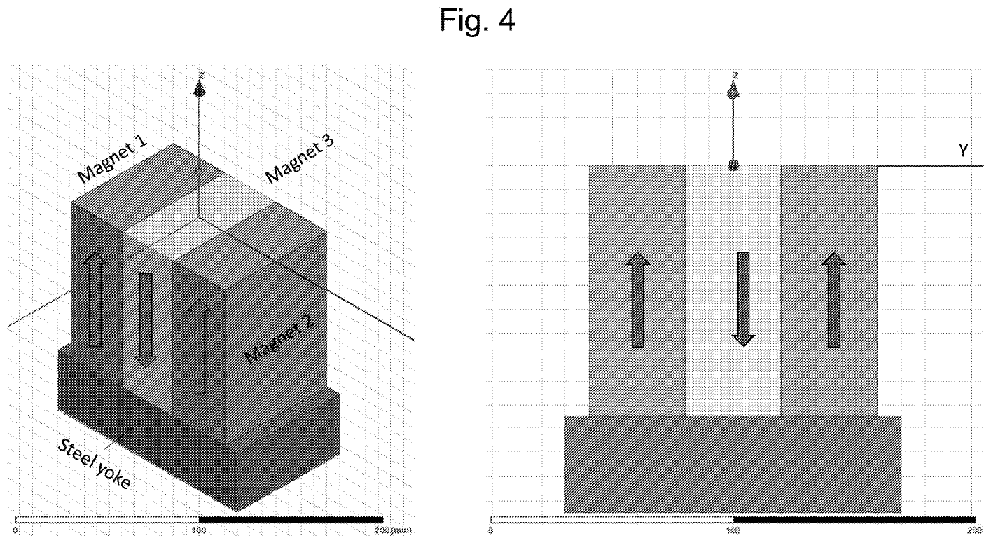

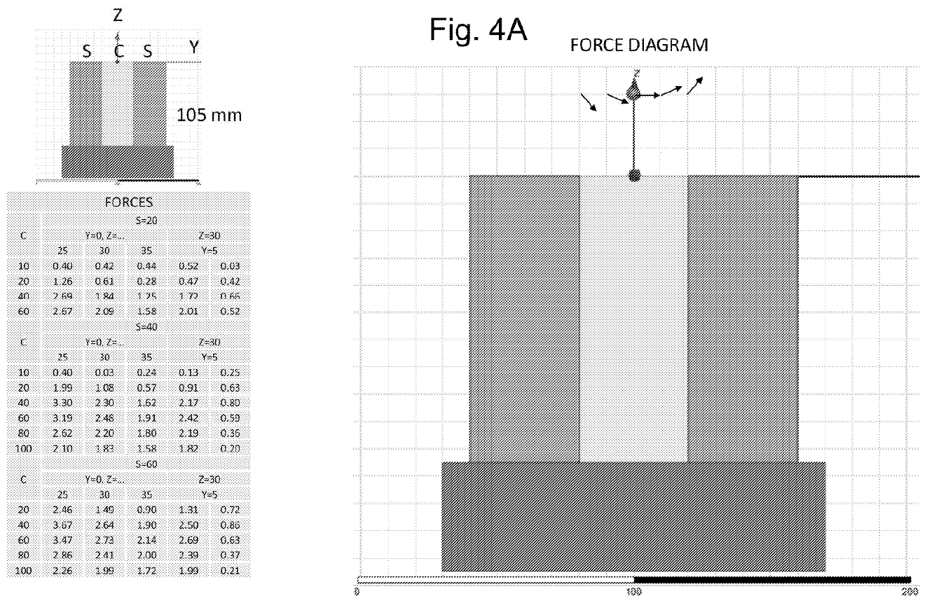

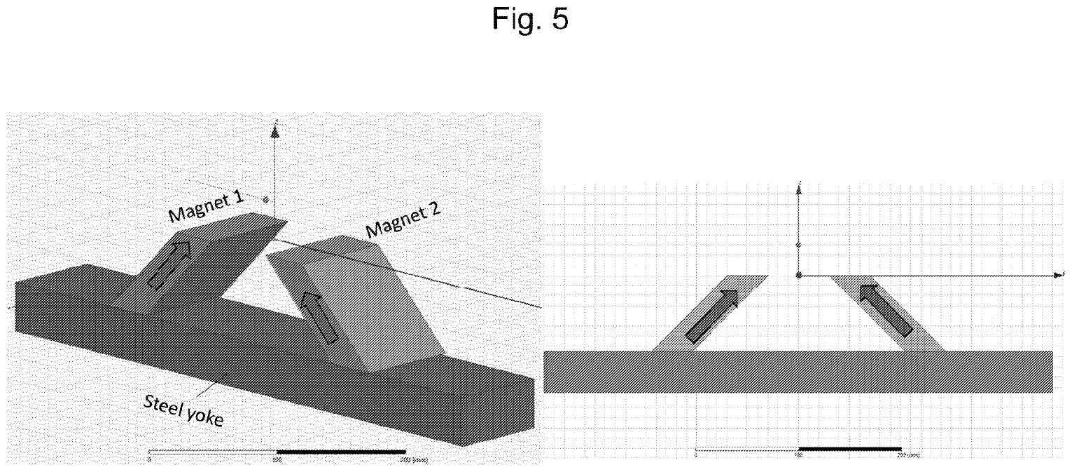

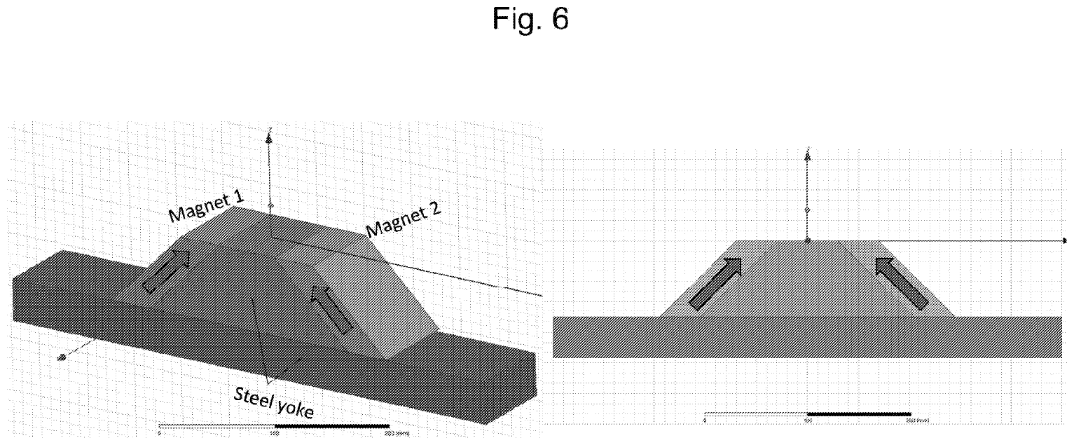

FIGS. 2, 3, 4, 5, 6, 7, and FIG. 8 illustrate various configuration of external magnetic systems, according to embodiments of the present invention. Dimensions, shapes, and materials used vary according to embodiment. In all these illustrations, internal devices are considered to have a horizontal magnetization along the y-axis.

FIGS. 2A, 3A, and 4A demonstrate the results of simulations estimating the force vectors operating on an internal device according to configurations illustrated in FIGS. 2, 3, and 4, respectively, for different values of the geometrical parameters of the system. As can be seen, the magnetic systems of embodiments of the invention provide variable control of the magnetic force vector in two dimensions (y-z) by varying the position of the internal device relative to the external magnetic system along the y-axis. This in turn provides variable degrees of horizontal versus vertical force and torque vectors on the internal device.

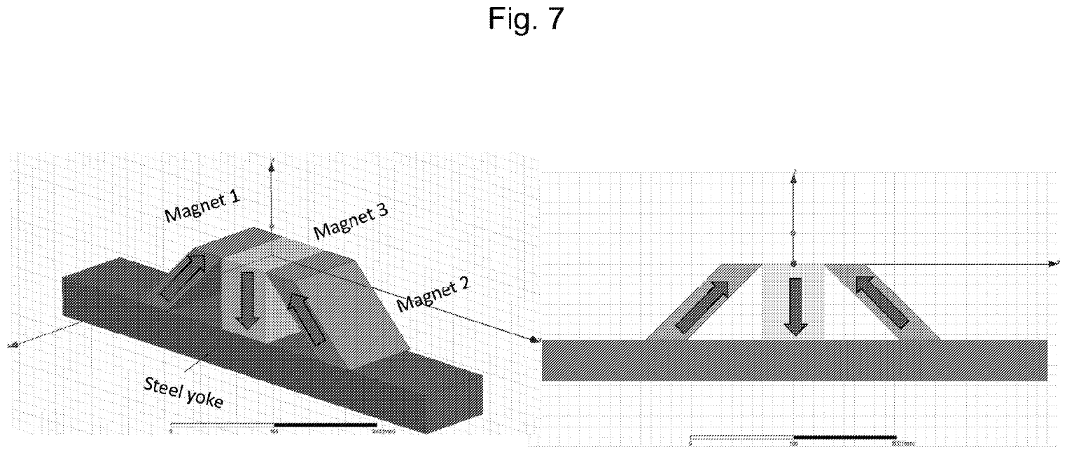

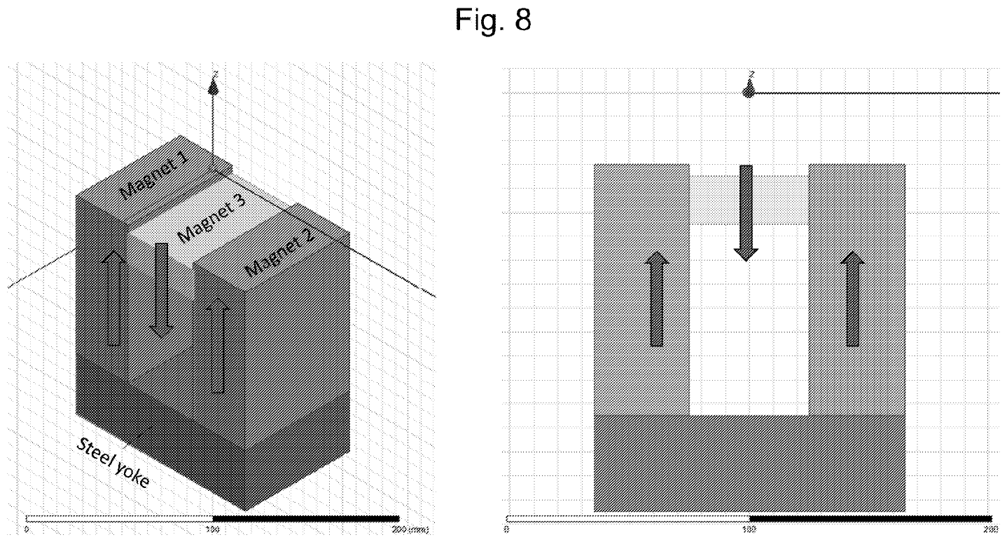

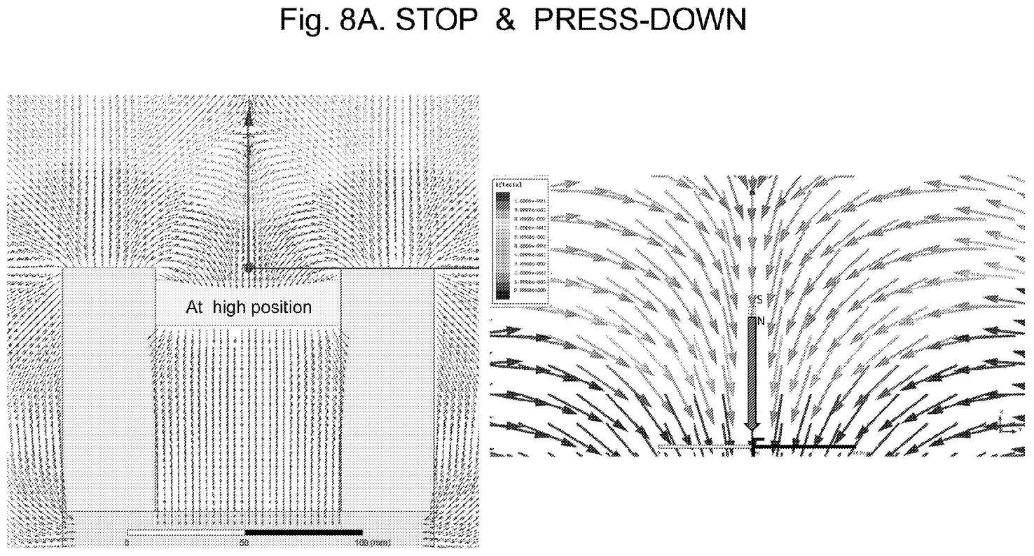

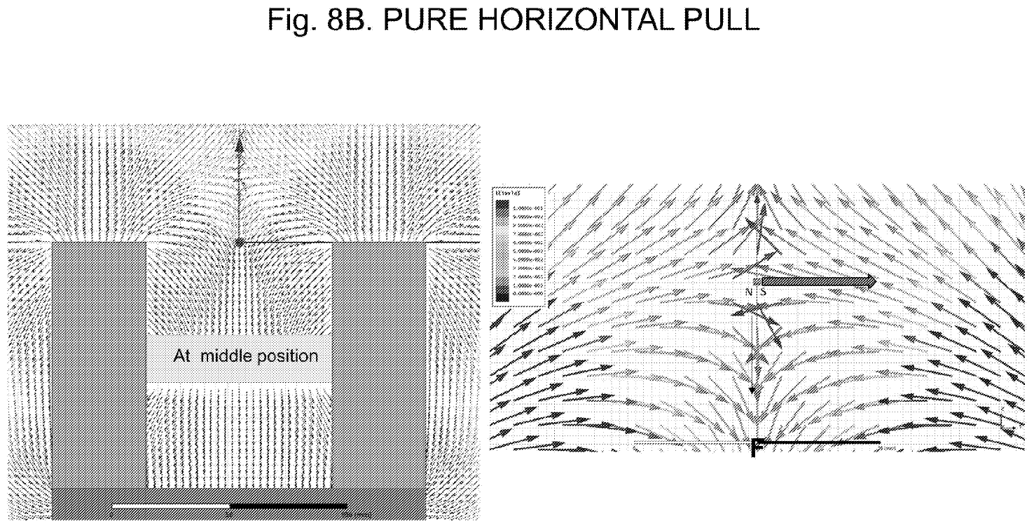

In other embodiments of the invention, variable torque/force vectors acting on the internal device can be generated by changing the internal configuration of the external magnetic system instead of or in addition to varying the position of the internal device with respect to the external magnetic system. FIG. 8 illustrates a configuration providing controlled motion of a subcomponent of the external magnetic system (Magnet 3) for adjusting the direction and strength of the magnetic field in the operational region where the internal device is located. FIG. 8A illustrates a related embodiment configuration which applies a pure pull force on the internal device towards the outer wall, thereby stopping it from moving. Changing the configuration of the external magnetic system to the one illustrated in FIG. 8B, results in the application of a purely horizontal force on the internal device.

According to a related embodiment, the direction of motion of an internal device can be reversed by changing the orientation of the external magnetic system components with respect to one another. In a non-limiting example, in FIG. 8 Magnets 1, 2, and 3 would be flipped vertically to achieve right-to-left motion. In another related embodiment, a length parameter C (FIG. 2) is adjusted to achieve right-to-left motion. In still another embodiment, this is done by changing the parameter dynamically; a further embodiment provides two different configurations for the external magnetic system, and alternates between them as needed, for left-to-right or right-to-left motion.

In another embodiment, the internal device within the lumen is flipped with respect to the external magnetic system, by applying a variable torque in the y-z plane.

As disclosed above, in various embodiments of the invention, the direction of the y-z force vector is accurately controlled by moving the external magnetic system along the y-axis with respect to the internal device. This provides dynamic control of the internal device motion based on a live feedback loop and changing of the direction of motion, to overcome irregularities, bifurcations, and so forth encountered in the lumen outer wall.

### Motion Along X-Axis

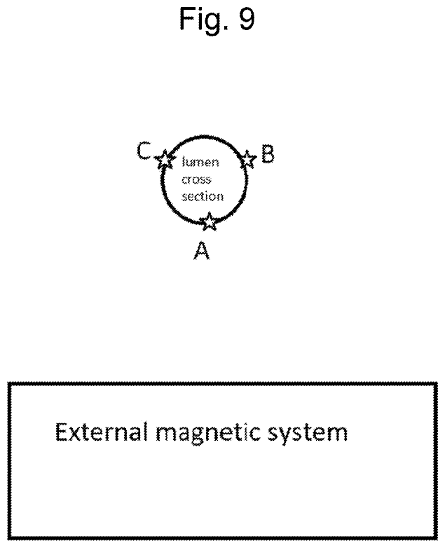

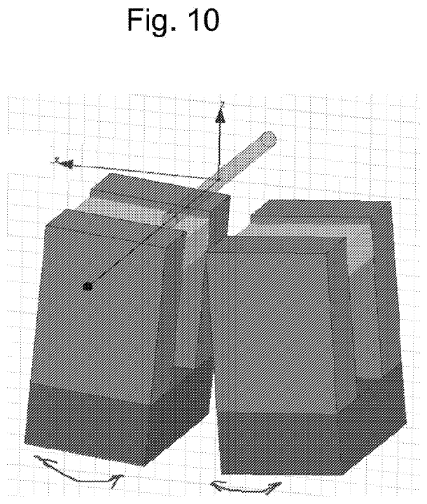

Under certain conditions, it may be desirable to have control of lateral motion along the x-axis inside the lumen. In a non-limiting example, FIG. 9 illustrates a cross-section of a lumen, with stars indicating possible desired locations of the internal device. The case above describes internal device motion at the A position (where the internal device is closest to the external magnet). In one specific scenario, it is desired to move the internal device to the B or C position, located 120° away from point A, as seen from the center of the lumen. According to one embodiment of the invention, this is achieved by moving the external magnetic system along the x-axis while rotating the magnet to face the internal device (see FIG. 10).

According to a further embodiment, changing the vector of force/torque in the y-z axis as disclosed above, causes the internal device to “climb” up the outer wall inside a lumen.

### Payload Release

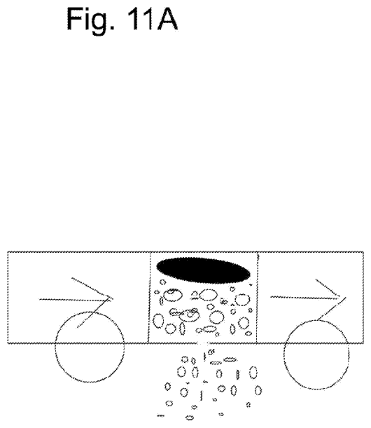

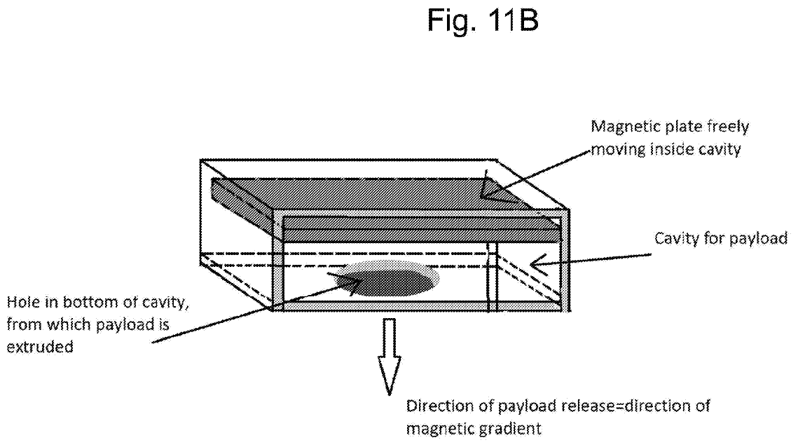

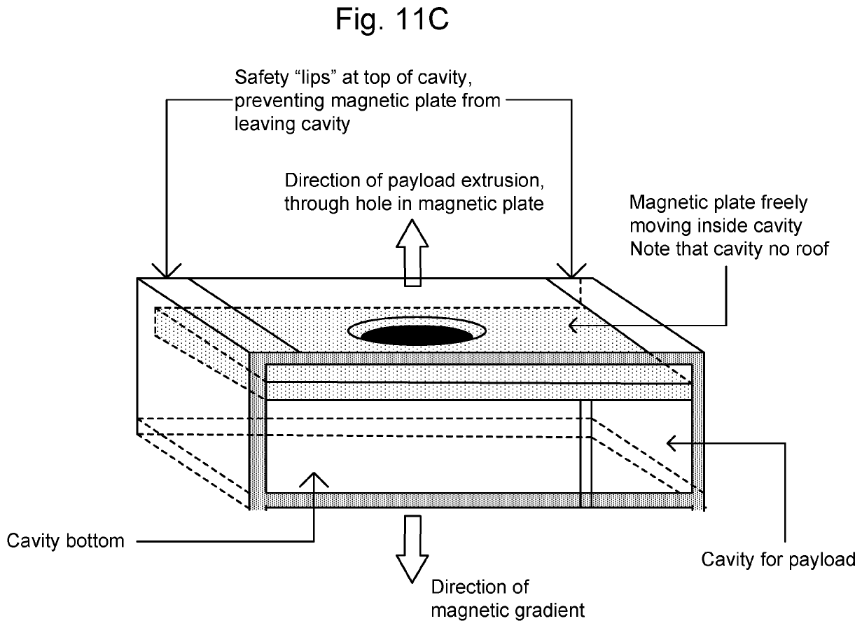

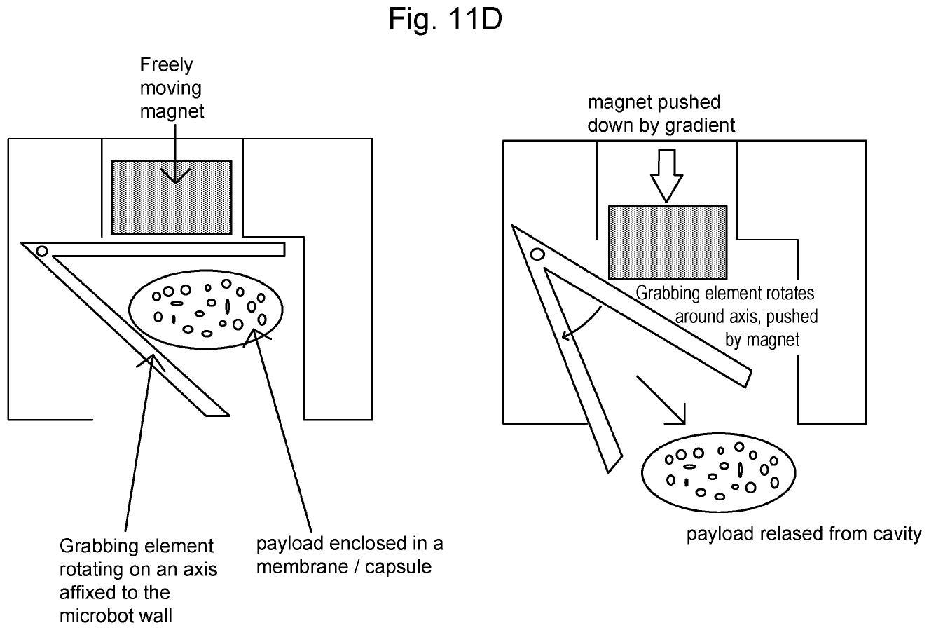

In certain medical applications it is desirable to release a payload from an internal device at a predefined location within a lumen via a remote-controlled trigger. Non-limiting examples of payloads include: therapeutic substances (e.g., drugs) and diagnostic aids (e.g., radio-isotopes, imaging contrast enhancement substances, etc.) Magnetic systems according to embodiments of the present invention provide payload release, by utilizing one or more cavities within an internal device which contain an embedded payload. FIG. 11A illustrates a configuration according to certain embodiments of the invention, wherein the cavity carrying the payload is embedded between two permanent magnetic compartments in the internal device, and the internal device is positioned on wheels touching a lumen outer wall. Inside the payload cavity there is a freely moving magnetic component (permanent magnet or ferromagnetic material), marked as a black oval in FIG. 11A. Such a component is denoted herein as a “magnetic piston.” When a vertical attractive force is applied by the external magnetic system on the internal device, the freely moving magnetic piston pushes the payload outside of the cavity, as schematically illustrated in FIG. 11A. In a related embodiment, the payload is encased in the cavity with an opening that is sealed by a membrane, and which is breached only when the pressure exerted on it from the magnetic piston is large enough to rupture the membrane.

According to certain embodiments of the invention, such a magnetic attractive force is generated by the same mechanism that stops the internal device; and the strength of this force is regulated by moving the external magnetic system closer to or further away from the internal device and/or manipulating the internal components of the external magnetic system, as illustrated in FIG. 8A and FIG. 8B.

In various embodiments of the invention, the force threshold for effective motion of the internal device in the lumen is herein defined as F₁, and the force threshold for payload release is herein defined as F₂.

It is important to prevent uncontrolled or unintended release of a payload during regular internal device motion. Therefore, certain embodiments of the invention utilize orthogonal vectors for linear motion and payload release, i.e., vectors F₁ and F₂ are orthogonal (such as by constraining linear motion to the y-axis while constraining payload release triggering to the x-z plane, according to FIG. 1). In certain other related embodiments, the system is configured so that the force magnitude threshold for payload release |F₂|>>|F₁|. In certain embodiments this is accomplished by minimizing F₁ (via reducing outer wall friction and lumen liquid content viscous resistance, such as with wheels, lubricating coating, and hydrodynamic streamlining) and/or increasing F₂ (such as by increasing rupture threshold of the membrane sealing the payload cavity, decreasing size of opening in cavity, or lowering the magnetic moment of the magnetic piston). In a related embodiment, payload release is accomplished in a single pulse of F₂ for total payload release at a single time; in another related embodiment, payload release is accomplished over the course of multiple pulses of F₂ for gradual or sequential payload release.

FIG. 11B and FIG. 11C schematically illustrate payload cavity configurations within the internal device, according to embodiments of the invention which provide payload release in different directions, not only in the direction towards the external magnetic system. This flexibility is desirable because the payload may need to be released towards a specific section of the lumen where a particular anatomical feature is located, rather than necessarily towards the external magnet. In a non-limiting example, it may be desired to release the payload towards the points B and/or C inside the lumen as illustrated in FIG. 9).

According to other embodiments, multiple mechanical variations on the same mechanism are provided, for pushing or expelling a liquid or gel payload, or depositing a solid payload from the cavity of the internal device. In a non-limiting example, FIG. 11D shows a mechanism for releasing a solid payload (a capsule) from the internal device, using a pulling force towards the external magnetic system.

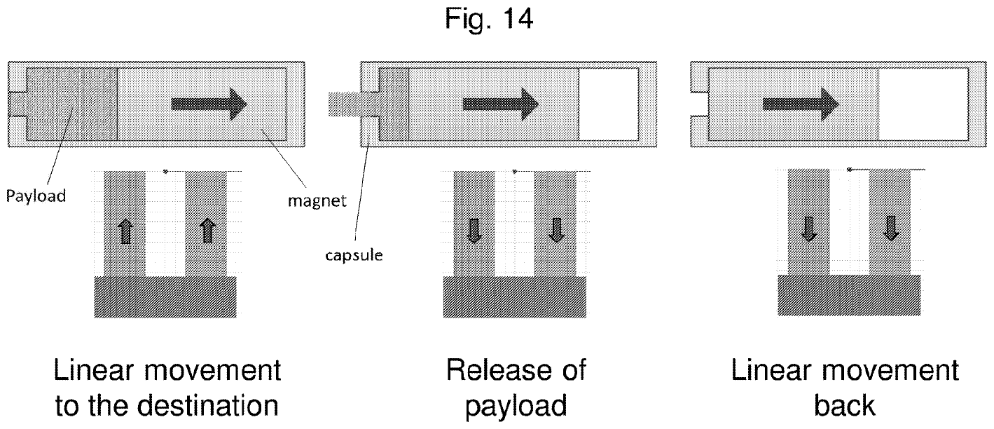

FIG. 14 illustrates an internal device, according to embodiments of the present invention, moving in one direction (right to left in this example) and that reverses direction upon reaching the target. In some embodiments, this is achieved by mechanically flipping the external magnet orientation (vertically in this example). In some embodiments, this is achieved by introducing a new external magnet with opposite (vertical in this example) magnetization. A piston inside the internal device immediately expels the payload and then the device begins to travel in the reverse direction (left to right in this example).

### Anchoring

In certain medical applications it is desirable to anchor an internal device to an outer wall of a lumen, for purposes including, but not limited to:
using the internal device that is clearly visible on a medical imaging system, as a fiduciary marker for a subsequent medical procedure;
timed and/or gradual release of a payload from the internal device;
monitoring a specific biochemical marker;
collecting data and/or detecting sensory activity;
enabling various types of treatment using the internal device as therapeutic instrument (e.g., thermal ablation, electrostimulation, radiotherapy); and
providing an anchor point for other medical components introduced to the lumen (e.g., a catheter).

Certain embodiments of the invention provide magnetic means (with an embedded permanent magnet or ferromagnetic component) for controllably anchoring an internal device to an outer wall of a lumen by applying an attractive force towards the outer wall which deploys an anchoring component that pierces the outer wall tissue and affixes the internal device to the outer wall. In accordance with other embodiments, the internal device offers multiple functionalities, including payload release and anchoring.

According to these embodiments, the force threshold for affixing the anchoring component is herein defined as F₃, and the force threshold for releasing the anchoring is approximated as −F₃. In a similar manner to previously-described embodiments, according to these present embodiments the system is configured such that |F₃|>>|F₁|, to guarantee that the anchoring will withstand linear motive forces and that there is no unintended anchoring during regular motion. Similarly, |F₂|>>|F₃|>>|F₁|, which supports the sequence of controlled motion, followed by controlled anchoring, followed by controlled payload release.

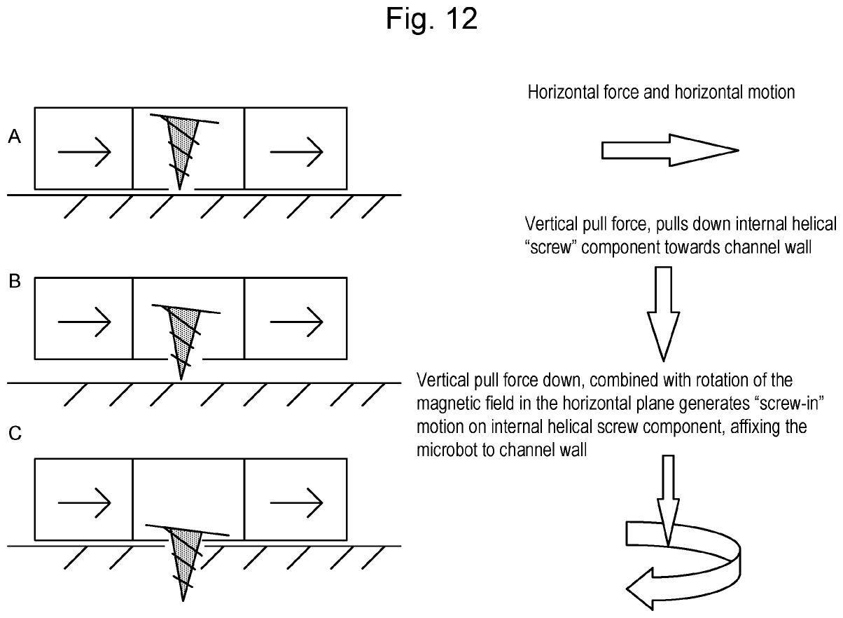

Certain embodiments of the invention provide a piercing screw motion for penetrating and affixing to the tissue of a lumen outer wall. FIG. 12 illustrates a non-limiting example of system where the piercing component has a helical shape, similar to that of a corkscrew or an aggressively-threaded dry-wall screw, facing an outer wall of the lumen (FIG. 12A). In a related embodiment the anchoring element is located inside the internal device between two permanent magnets having fixed horizontal magnetization. The anchoring component is magnetic, as described above. In embodiments where a permanent magnet is used, it is magnetized radially (i.e., orthogonal to the screw's axis of symmetry). As the attractive force towards the outer wall is generated, the anchoring component emerges from the internal device, pushes against the outer wall, and penetrates it (FIG. 12B). At the same time, the external magnetic system is rotated in a plane tangent to the outer wall, thereby inducing a rotational torque on the anchoring element around its axis of symmetry, in addition to the attractive force along the axis of symmetry, effectively screwing it into the outer wall of the lumen and thereby anchoring the internal device to the inside of the outer wall.

According to certain embodiments, the anchoring procedure is reversible, by applying a repelling force on the internal device away from the lumen outer wall and rotating the external magnetic system in the opposite direction, thereby providing a releasably-anchorable internal device.

Internal Device with Magnetic Elements Interconnected by Non-Magnetic Flexible Connectors

According to embodiments of the present invention, the internal device is comprised of a set of magnetic elements connected by non-magnetic flexible connectors. The elements can have multiple functions, such as device motion, payload release, or anchoring to a lumen wall.

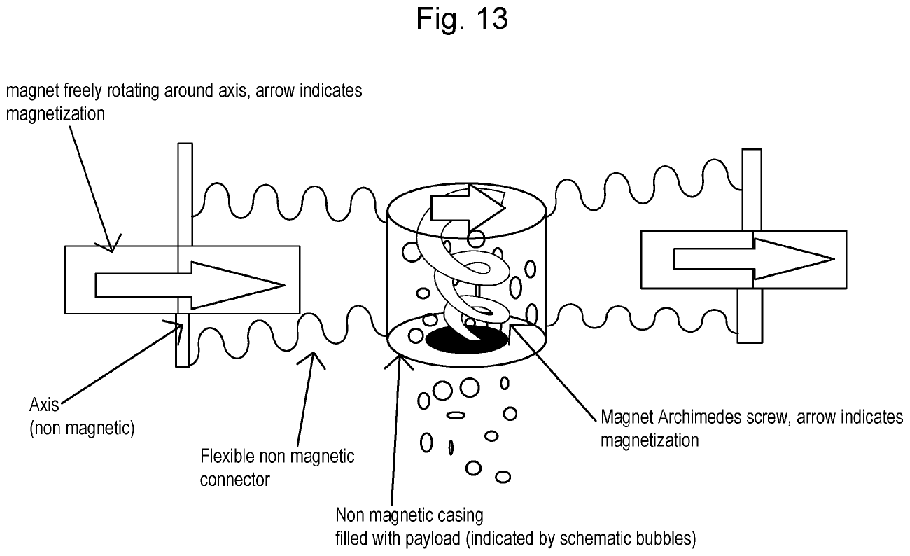

FIG. 13 depicts an internal device with two magnetic components and a payload release element in between them, moving along the y axis in this example. The magnetic component is comprised of a permanent magnet freely rotating around a non-magnetic axis. The rotation axis in this example is parallel to the z-axis (i.e., orthogonal to lumen wall). The magnetization of the permanent magnet is in the y direction of motion, by definition. A payload carrier is comprised of a non-magnetic casing filled with payload, with a built-in “archimedes screw” or feed screw, facing a hole in the wall of the casing. The magnetization of the screw is radial, in x-y plane. During motion of the device in the y direction, all magnetic components are aligned in the direction of the y-axis. When the device stops the external magnetic field begins to rotated in the x-y plane. In this example, each magnetic component rotates around its axis (instead of the entire device rotating around its axis). Because the lumen is narrow, this method prevents the device from getting stuck across the lumen. At the same time, the payload release element contains a radially magnetized screw facing a hole in the casing wall. As the screw rotates it expels payload to the outside of the casing.

Note that for all motion of the device, the forces and torques operating on the device components are roughly the same. So they will move in unison.

The distances between neighboring elements in this configuration should be maintained above a certain minimal threshold, to ensure the different magnetic elements do not influence each other (i.e., stick together or exert torque on each other). This can be achieved by using a non-magnetic elastic spring as a connector between neighboring elements.

In some embodiments, instead of a payload release element there is an anchoring element with an actual screw (not a feed screw), allowing anchoring of the element to the lumen wall.

In some embodiments, the rotating external magnetic field has no magnetic field gradient in the direction of the axis of the rotating magnetic field. In some embodiments, the rotating external magnetic field has a magnetic field gradient in the direction of the axis of the rotating magnetic field for exerting a force on the screw along the axis of the rotating magnetic field. In some embodiments, the screw mechanism can be used in reverse rotational direction, to “suck in” elements from the outside to the inside or to allow the screw to reversibly detach from the lumen wall.

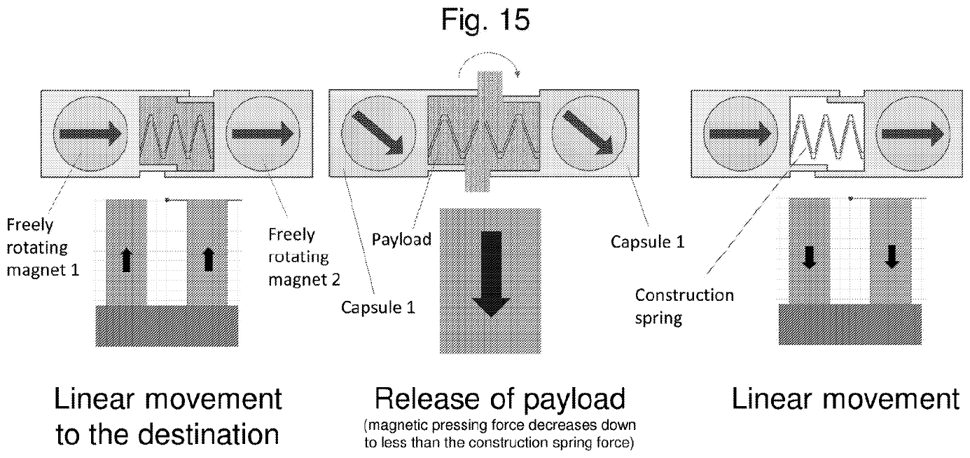

FIG. 15 depicts an internal device, according to embodiments of the present invention, with two magnetic components and a cavity in between them, moving along the y-axis in this example. The two magnetic components each contain a freely rotating round magnet inside it, with radial magnetization. The opposite poles of the two magnets face each other to generate an attracting force. The two magnetic components are separated by a cavity. Inside the cavity there is a payload and a flexible non-magnetic spring that pushes the two magnetic components apart. However, in the case of motion in the y direction (parallel to lumen), the opposing poles of the two magnets inside the two magnetic components are attracted to each other, and they generate a force stronger than the force of the spring repelling the magnetic components. As a result, the two magnetic components remain close to each other, preventing the payload from escaping the cavity. When the direction of the magnetic field is changed (down along the z-axis in this example), the freely rotating magnetic elements in the two magnetic components rotate (to face down in this example). Now their similar poles are close to each other, and therefore generate a repulsing force instead of an attracting force. Consequently, the magnetic components push each other away and the cavity between them opens, thereby releasing the payload. When the magnetic field turns to the left (reversing the direction of motion of the device) or to the right (continuing along the direction of motion of the device), the internal device returns to its initial configuration.

It should be noted that the internal device would be subject to both varying forces and varying torques, which in turn could change the orientation of the internal magnet embedded in the internal device inside the lumen. If the magnetic component of the internal device is freely rotating (e.g., a spherical magnet freely rotating in a shell), this means this external shell would not rotate, while the magnet rotates until an equilibrium position where torque acting upon it equals zero. If the internal device's shell is affixed to the internal magnet, then the torque would act upon the entire device. Both situations could be utilized according to the circumstances. For instance, if application of a specific force on the internal device (irrespective of device orientation in the lumen) is desired, the device may be provided with a freely rotating magnet inside it. Alternatively, if a change in the spatial orientation of the device (e.g., to point the device diagonally) is desired, the internal magnet may be affixed to the device.

It should also be noted that the instantaneous force acting on a freely rotating internal magnet would be different from the equilibrium force acting upon on the same magnet (i.e., the force operating on the magnet while the torque is zero). This is true because initially the internal device may not be at its equilibrium position. When the torque acting upon the freely rotating internal magnet is zero, the angle of the internal magnet in reference to the y-plane is denoted as the equilibrium angle.

The spatial distribution of the magnetic field (force and torque) generated by the external magnetic system is non uniform in the lumen. As a result, the equilibrium force and equilibrium angle will be different at each point in the lumen. By definition, when a device is located at point A relative to the external magnetic system and is moving in reference to the external magnetic system, it will instantaneously move to a new point B, where the force and torque acting upon it would be different than at point A. By carefully designing the magnetic system, a specific spatial distribution of forces and torques can be created which allows better motion control of the internal magnet. Of particular interest are force/torque distributions that ensure the internal magnet does not diverge from its desired motion trajectory.

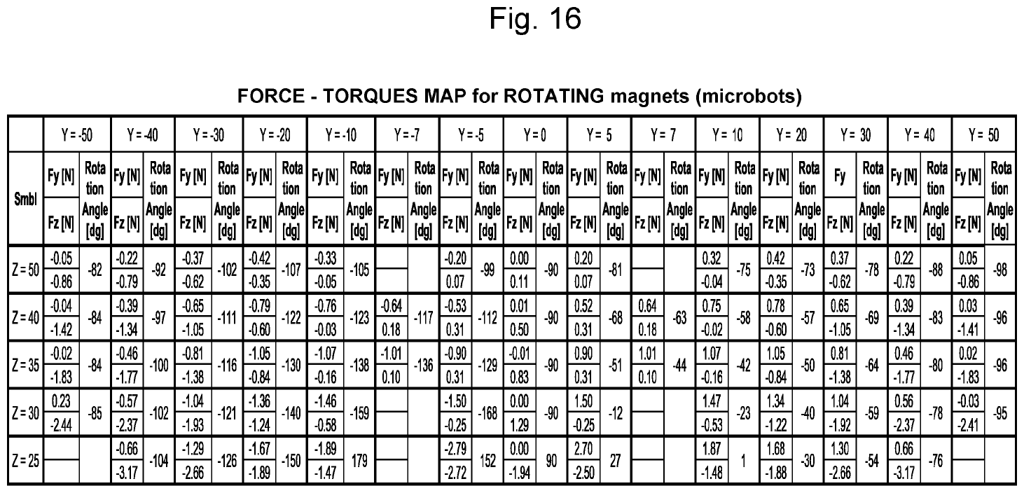

For example, FIG. 16 shows the distribution of equilibrium force and equilibrium angle, for a particular embodiment of the magnetic system corresponding to FIG. 8. Forces are in mN and distances are in mm. In this specific configuration, negative horizontal force can be achieved in areas where Y<0 (assume Y=0 at the symmetry axis of the external magnetic system) and positive horizontal force can be achieved where Y>0. This allows effective bidirectional horizontal motion control Also, it should be noted that as Y grows in this particular configuration, the Z component of the equilibrium force becomes strongly negative while the Y component goes closer to zero, effectively attaching the device to the lumen wall closest to the external magnet and preventing device divergence (i.e., a negative feedback loop in control theory nomenclature). This makes motion control of the device more reliable. The same phenomenon is seen symmetrically in the Y<0 region.

It should be noted that as the distance between the internal magnet and the magnetic control system goes down, the Z force component becomes strongly negative, while the Y component is close to zero. This allows efficient control of the Z motion and prevents drifting of the device away from the lumen surface closest to the control system.

It should be noted that the equilibrium angle near the Y=0 line is circa −90 degrees. This means that the freely rotating magnet would be oriented orthogonally relative to the lumen wall. This again highlights why it is important to have a freely rotating magnet in this configuration, as otherwise the microbot may be oriented orthogonally to the lumen wall, which may be undesired.

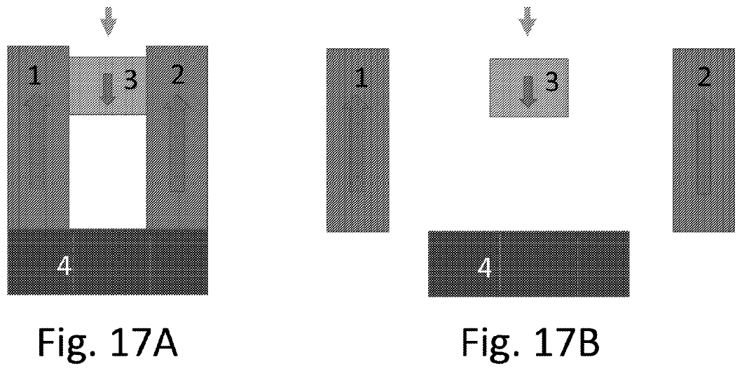

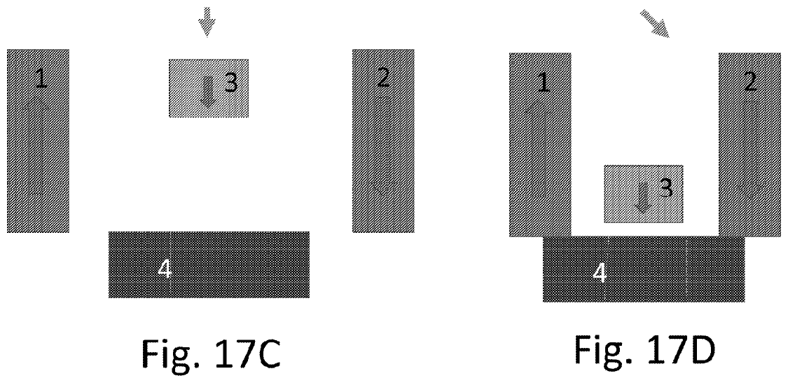

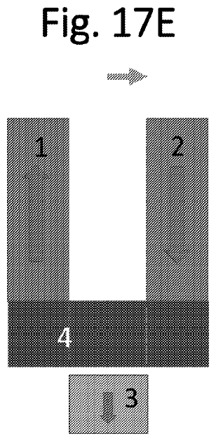

In certain scenarios, it may be desirable to reposition the freely rotating magnet so it is oriented horizontally (i.e., equilibrium degree of 0 an not −90) and then rotate it in the x-y plane for various activities. Such activities may include anchoring the device to the lumen wall (see FIG. 12) or expelling a payload from a device using a feed screw mechanism (see FIG. 13). FIGS. 17A-17D show how this can be achieved by mechanically manipulating the configuration of the external magnetic control system.

FIG. 17A shows an initial configuration corresponding to FIG. 8, with 4 external magnetic components indicated by the numbers 1, 2, 3, and 4, respectively. The large arrows indicate the magnetization direction of magnets 1-3. The green arrow above the external magnetic system indicates the equilibrium angle of the internal magnet inside the lumen (−90 degrees initially). The white dotted lines in component 4 (the yoke) indicate a hole allowing mechanical motion of component 3 (the middle magnet) through component 4.

To start, magnets 1 and 2 are mechanically moved to the side, thereby keeping the internal magnet in place by the gradient generated by magnet 3 (see FIG. 17B). Next, magnet 2 is rotated in the x-z plane, flipping its magnetization vertically. This is now easier to accomplish mechanically as magnet 2 is distanced from other system components (minimizing forces and torques) (see FIG. 17C).

Next, magnet 3 is pushed down through the hole in the yoke 4, while simultaneously bringing magnets 1 and 2 closer to their initial position. Now the internal magnet is more affected by magnets 1 and 2 and not by magnet 3. As a result, the equilibrium angle changes (see FIG. 17D).

In the final configuration (FIG. 17E), magnet 3 is located below yoke 4 and exerts no force on the internal magnet. The internal magnet is pulled down towards the external magnetic system, and its equilibrium angle is 0 as desired. In this situation, the external magnetic control system is rotated in the x-y plane as needed. Note that the process is reversible (i.e., we can switch back to configuration 17 A as needed).

What is claimed is:

1. A device for magnetically controlling a target object within a lumen, the device comprising:
at least two permanent dipole magnets of like dimensions and corresponding like faces, and having like poles disposed on respective like faces thereof;
a yoke incorporating a material capable of completing a magnetic circuit;
wherein the at least two permanent dipole magnets are affixed to the yoke adjacent to one another such that:
like pole faces of the at least two permanent dipole magnets are affixed in contact with a same single face of the yoke;
the at least two permanent dipole magnets are affixed to the yoke in locations on the same single face of the yoke such that an inter-magnet space separates the at least two permanent dipole magnets; and
respective opposite pole faces of the at least two permanent dipole magnets are exposed, adjacent, and separated by the inter-magnet space.

2. The device of claim 1, wherein magnetically controlling the target object includes controlling at least one of:
a spatial position of the target object; and
a spatial angular orientation of the target object.

3. The device of claim 1, wherein the material capable of completing a magnetic circuit includes at least one of:
a paramagnetic material; and
a ferromagnetic material.

4. The device of claim 1, wherein the at least two permanent dipole magnets include at least one permanent magnet that is interchangeably replaceable by a permanent magnet selected from a plurality of permanent magnets having different magnetic field strengths.

5. The device of claim 4, wherein the plurality of permanent magnets having different magnetic field strength includes at least one magnet selected from a group consisting of:
a permanent magnet for controlling motion of the target object; and
a magnet for controlling release of a payload from the target object.

6. The device of claim 1, wherein the at least two permanent dipole magnets include at least one permanent magnet that is interchangeably replaceable by a permanent magnet having a predetermined magnetic field orientation.

7. The device of claim 6, wherein the predetermined magnetic field orientation is chosen from a group consisting of:
an orientation for controlling motion of the target object in a predetermined direction; and
an orientation for controlling release of a payload from the target object.

8. The device of claim 1, wherein the at least two permanent dipole magnets are geometrically congruent.

9. The device of claim 8, wherein a shape of the at least two permanent dipole magnets is selected from a group consisting of:
a prism;
an anti-prism;
a cylinder; and
a truncated cone.

10. The device of claim 1, wherein a length dimension of the at least two permanent dipole magnets is a distance between opposite magnetic poles of a permanent dipole magnet.

11. The device of claim 10, wherein the length dimension is the largest dimension of the at least two permanent dipole magnets.

12. The device of claim 11, wherein the length dimension is substantially greater than the inter-magnet space.

13. The device of claim 1, further comprising yoke extension incorporating a material capable of completing a magnetic circuit, wherein a face of the yoke extension is affixed to the same single face of the yoke and is disposed in the inter-magnet space.

14. The device of claim 1, wherein respective pole orientations of the at least two permanent dipole magnets are parallel, and further comprising at least one opposing permanent dipole magnet disposed in the inter-magnetic space, wherein the opposing permanent dipole magnet is oriented such that the pole orientation thereof is anti-parallel to that of the at least two permanent dipole magnets.

15. The device of claim 14, wherein a pole of the opposing permanent dipole magnet nearest the yoke is in contact with the yoke.

16. The device of claim 14, wherein a pole of the opposing permanent dipole magnet nearest the yoke is at a variable distance from the yoke.

17. The device of claim 1, wherein the at least two permanent dipole magnets are oblique and are disposed such that a minimum distance between their like faces affixed to the yoke is substantially greater than a minimum distance between their exposed like faces.

18. The device of claim 17, further comprising at least one opposing permanent dipole magnet disposed in the inter-magnetic space, wherein the opposing permanent dipole magnet is oriented such that the pole orientation thereof is opposite to that of the at least two permanent dipole magnets.

19. The device of claim 1, wherein the target object comprises wheels for orienting the target object with respect to an interior wall of the lumen.

20. The device of claim 1, wherein magnetically controlling the target object includes controlling the release of a payload from a cavity in the target object into the lumen.

21. The device of claim 20, wherein the target object comprises wheels for orienting the target object with respect to an interior wall of the lumen.

22. The device of claim 20, wherein the target object further includes a piston incorporating a magnetic material, wherein the piston is operative to expel the payload from the cavity when subjected to a magnetic field from the device.

23. The device of claim 22, wherein the cavity includes an aperture through which the payload is expelled from the cavity into the lumen.

24. The device of claim 22, wherein the piston includes an aperture through which the payload is expelled from the cavity into the lumen.

25. The device of claim 23, wherein the target object further includes a membrane over the aperture, wherein the membrane is configured to rupture at a predetermined rupture pressure, thereby expelling the payload from the cavity into the lumen when the piston exerts a pressure at least as great as the predetermined rupture pressure.

26. The device of claim 24, wherein the target object further includes a membrane over the aperture, wherein the membrane is configured to rupture at a predetermined rupture pressure, thereby expelling the payload from the cavity into the lumen when the piston exerts a pressure at least as great as the predetermined rupture pressure.

27. The device of claim 13, further comprising:
a pivotable scoop within the cavity, for manipulating the payload; and
an actuator therefor;
wherein:
the pivotable scoop, when closed inside the cavity by the actuator, retains the payload in the cavity;
the pivotable scoop, when opened from the cavity by the actuator, expels the payload from the cavity into the lumen; and
the actuator incorporates a magnetic material operational to pivot the pivotable scoop when subjected to a magnetic field from the device.

28. The device of claim 1, wherein magnetically controlling the target object includes controlling an anchor within the target object for affixing the target object to an interior wall of the lumen.

29. The device of claim 28, wherein the anchor of the target object includes:
a captive penetrating screw member for penetrating and screwing into an interior wall of the lumen, thereby affixing the target object to the interior wall of the lumen; and
a screw actuator incorporating a permanent magnet operative to apply a torque to the captive penetrating screw member when subjected to a rotating magnetic field from the device, such that the captive penetrating screw member screws into the interior wall of the lumen, thereby affixing the target object to the interior wall of the lumen.

30. The device of claim 29, wherein the screw actuator is incorporated directly into the captive penetrating screw member.

31. The device of claim 29, wherein the screw actuator is further operative to apply a reverse torque to the captive penetrating screw member when subjected to a reverse rotating magnetic field from the device, such that the captive penetrating screw member unscrews from the interior wall of the lumen, thereby releasing the target object from the interior wall of the lumen.

32. The device of claim 29, wherein the rotating magnetic field from the device has no magnetic field gradient in a direction of an axis of the rotating magnetic field.

33. The device of claim 30, wherein the rotating magnetic field from the device has no magnetic field gradient in a direction of an axis of the rotating magnetic field.

34. The device of claim 31, wherein the rotating magnetic field from the device has no magnetic field gradient in a direction of an axis of the rotating magnetic field.

35. The device of claim 29, wherein the rotating magnetic field from the device has a magnetic field gradient in a direction of an axis of the rotating magnetic field for exerting a force on the screw actuator along the axis of the rotating magnetic field.

36. The device of claim 30, wherein the rotating magnetic field from the device has a magnetic field gradient in a direction of an axis of the rotating magnetic field for exerting a force on the screw actuator along the axis of the rotating magnetic field.

37. The device of claim 31, wherein the rotating magnetic field from the device has a magnetic field gradient in a direction of an axis of the rotating magnetic field for exerting a force on the screw actuator along the axis of the rotating magnetic field.

38. The device of claim 5, wherein:
the target object comprises a screw actuator for releasing the payload;
wherein the screw actuator incorporates a permanent magnet operative to apply a torque to the screw actuator when subjected to a rotating magnetic field from the device.

39. The device of claim 38, wherein the rotating magnetic field from the device has a magnetic field gradient in a direction of an axis of the rotating magnetic field for exerting a force on the screw actuator along the axis of the rotating magnetic field.

40. An internal device for implanting into a biological medium and for responding to control by an external magnetic control system, the internal device comprising a plurality of linearly-interconnected magnetic elements along a device axis, such that each magnetic element of the plurality is adjacently-connected to at least one other element and at most two other elements, wherein:
each pair of adjacent magnetic elements is interconnected by a non-magnetic flexible connector; and
each magnetic element of the plurality has at least one predetermined function selected from a group consisting of:
a function relating to control by the external magnetic control system; and
a function relating to data communication with the external magnetic control system.

41. The internal device of claim 40, wherein a function relating to control by the external magnetic control system is selected from a group consisting of:
a function relating to linear motion of the internal device within the biological medium;
a function relating to angular motion of the magnetic element;
a function relating to angular motion of the internal device;
a function relating to a connection of the magnetic element to an adjacently-connected magnetic element;
a function relating to anchoring the internal device to a feature within the biological medium;
a function relating to releasing an anchoring of the internal device from a feature within the biological medium; and
a function relating to a release of a payload carried by the magnetic element.

42. The internal device of claim 40, wherein a function relating to data communication with the external magnetic control system is selected from a group consisting of:
receiving data from the external magnetic control system relating to control of the magnetic element;
receiving data from the external magnetic control system relating to control of the internal device;
transmitting data to the external magnetic control system relating to a status of the magnetic element;
transmitting data to the external magnetic control system relating to position of the internal device within the biological medium;
transmitting data to the external magnetic control system relating to an anchoring of the internal device to a feature within the biological medium;
transmitting data to the external magnetic control system relating to a releasing of an anchoring of the internal device from a feature within the biological medium;
transmitting data to the external magnetic control system relating to angular orientation of the magnetic element within the biological medium;
transmitting data to the external magnetic control system relating to angular orientation of the internal device within the biological medium;
transmitting data to the external magnetic control system relating to velocity of the internal device within the biological medium;
transmitting data to the external magnetic control system relating to angular velocity of the magnetic element within the biological medium;
transmitting data to the external magnetic control system relating to angular velocity of the internal device within the biological medium;
transmitting data to the external magnetic control system relating to a payload carried by the magnetic element; and
transmitting data to the external magnetic control system relating to a payload released by the magnetic element.

43. The internal device of claim 40, wherein:
at least one magnetic element of the plurality has a predetermined function relating to a motion; and
at least one magnetic element of the plurality has a predetermined function relating to a release of a payload.

44. The internal device of claim 41, wherein the predetermined function relates to a motion, and wherein:
the magnetic element comprises a permanent magnet with a magnetization axis; and
the permanent magnet is operative to rotate around a rotation axis which is orthogonal both to the device axis and to the magnetization axis.

45. The internal device of claim 40, wherein a magnetic element is operative to rotate about the device axis independent of a rotation about the device axis of other magnetic elements.

46. The internal device of claim 41, wherein the predetermined function relates to a release of a payload carried by the magnetic element, and wherein the magnetic element includes a radially-magnetized feed screw operative to expel the payload.

47. A device for magnetically controlling a target object within a lumen, the device comprising:
at least two permanent dipole magnets of like dimensions and corresponding like faces, and having like poles disposed on respective like faces thereof;
a yoke incorporating a material capable of completing a magnetic circuit and having a passage through the yoke from an opening in a first face of the yoke to an opening on an opposing face of the yoke;
wherein the at least two permanent dipole magnets are reversibly affixed to the yoke adjacent to one another such that:
like pole faces of the at least two permanent dipole magnets are reversibly affixed in contact with a same single face of the yoke;
the at least two permanent dipole magnets are reversibly affixed to the yoke in locations on the first face of the yoke such that an inter-magnet space separates the at least two permanent dipole magnets;
respective opposite pole faces of the at least two permanent dipole magnets are exposed, adjacent, and separated by the inter-magnet space
a moveable opposing permanent dipole magnet disposed in the inter-magnetic space and capable of traversing the passage through the yoke.

48. The device of claim 47, wherein respective pole orientations of the at least two permanent dipole magnets are parallel, and the opposing permanent dipole magnet is oriented such that the pole orientation thereof is anti-parallel to that of the at least two permanent dipole magnets.

49. The device of claim 47, wherein respective pole orientations of the at least two permanent dipole magnets are anti-parallel.

50. The device of claim 47, wherein the at least two permanent dipole magnets are slidable along the first face of the yoke.

51. The device of claim 47, wherein at least one of the two permanent dipole magnets is configured to rotate, thereby flipping its pole orientation.
